(19) United States
(12) Patent Application Publication
(10) Pub. No.: US 2005/0233949 A1
(43) Pub. Date: Oct. 20, 2005

(54) Conjugates comprising cancer cell specific ligands, a sugar and cancer chemotherapeutic agents/boron neutron capture therapy agents, and uses thereof

(76) Inventors: Michael F Holick, Sudbury, MA (US); Halasya Ramanathan, Mysore (IN)

Correspondence Address: STERNE, KESSLER, GOLDSTEIN & FOX PLLC, 1100 NEW YORK AVENUE, N.W., WASHINGTON, DC, 20005, US

(21) Appl. No.: 10/510,827

(22) PCT Filed: Apr. 14, 2003
(86) PCT No.: PCT/US03/11374

§ 371 Date: Oct. 15, 2004

(30) Foreign Application Priority Data
Apr. 12, 2002 (US) 60371674

(51) Int. Cl. A61K 38/17; A61K 31/70; A61K 31/7048; A61K 31/7072; A61K 31/69; A61K 31/337

(52) U.S. Cl. 514/8; 514/27; 514/49; 514/449; 514/23; 514/64; 530/322

(57) ABSTRACT

Disclosed are conjugates comprising cancer cell specific ligands, a sugar and cancer chemotherapeutic agents/boron neutron capture therapy agents, and uses thereof, e.g. for treating or ameliorating hyperproliferative diseases.

### BACKGROUND OF THE INVENTION

1. Field of the Invention

The present invention relates to conjugates comprising a cancer cell specific ligand, linked to a sugar, linked to a cancer chemotherapeutic agent or or boron neutron capture therapy (BNCT) agent and uses thereof.

2. Related Art

The major problem associated with cancer chemotherapy is the high toxicity of the chemotherapeutic agents. This is a problem general to most of the chemotherapeutic agents including taxol, daunorubicin, cisplatin fluorouracil, vincristine, etoposide and others. Since cancer chemotherapeutic agents do not target cancer cells exclusively, toxicity cannot be avoided.

Toxicity is also apparent for other methods of treating cancer such as photodynamic therapy (PDT) and BNCT. In photodynamic therapy and boron neutron capture therapy, if the passive drug has no specificity, damage to the neighboring cells is inevitable when the drug is activated by either light or neutron capture. Similarly, diagnosis of cancer and monitoring the treatment progress of cancer patients using magnetic resonance imaging and contrast agents give poor target specificity.

Specific targeting of cancer cells has been attempted with antibody tethered liposomes. However, a problem with this approach is that the antibody should be tethered away from the huge liposomal molecule containing the cancer drug (without the spill), so that the target specificity can be enhanced.

Etoposide is a glucose-derivative of naturally occurring epipodophyllotoxin. It has been shown that epipodophyllotoxin-glucoside is also an effective cancer chemotherapeutic agent. This is believed to be due to the lack of water solubility of podophyllotoxin. The same approach has been used for camptothecin derivatives.

A large number of drugs and their metabolites are conjugated in the body as part of the elimination pathway. Glucuronic acid is the most frequent partner to the drug in conjugation. Remington's Pharmaceutical Sciences, A. Osol et al. (eds.), pp. 677 (1980).

ER (estrogen receptor), PR (progesterone receptor), and Her-2 receptors are over expressed in cancer cells. Peptides specific for ER have been prepared using a combinatorial peptide phage-display library for the preparation of ER-specific mAbs. S. Sompuram, et al., Clin. Chem. 48: 410-420 (2002).

The urokinase-type plasminogen activator (uPA) system is strongly linked to pathological processes, such as cell invasion and metastasis in cancer Dano K et al., Adv. Cancer Res. 44:139-266 (1985)). Cancer cells exhibit up-regulation of uPAR, a cell surface receptor. Tapiovaara H. et al., Adv. Cancer Res 69:101-33 (1996).

U.S. Pat. No. 6,120,765, discloses purified uPA peptides, having mitogenic activity and containing at least 6 amino acids of the EGF-like domain of uPA. These peptides bind to the urokinase plasminogen activator receptor (uPAR) on cells. Also described a conjugates between the uPA peptides and toxins, e.g. peptide toxins, for delivery to cells, e.g. epidermal cells.

U.S. Pat. No. 6,277,818 discloses uPAR targeting cyclic peptides comprising 11 amino acids corresponding to uPA (20-30), and substitution variants thereof. These cyclic peptides may be conjugated to diagnostic labels or therapeutic radionuclides. The compounds are reportedly useful for targeting uPAR expressed in pathological tissues, e.g. tumor cells. See also U.S. Pat. No. 5,942,492, which discloses cyclic peptides having 11 amino acids joined by a linking unit L, which are useful for inhibiting the growth or metastasis of cancerous tumors.

U.S. Pat. No. 6,258,360 discloses prodrugs of cytotoxic chemotherapeutic agents which are stable to endogenous mammalian enzymes and which are activated by tumor-specific antibody bonding and prodrug activation. The antibodies are capable of cleaving the protective moiety from the drug by esterase, amidase, hydrolase or glycosidase activity.

According to the present invention, by linking a cancer cell targeting agent to a sugar residue linked to a cancer chemotherapeutic agent/BNCT agent containing phenolic, hydroxy, carboxyl or enolizable functional groups, one obtains a pro-drug that offers many advantages. First and foremost, the cancer cell targeting agent targets the cancer cell, thus reducing toxicity to non-target tissues and cells. The bond between the agent and sugar is then cleaved iii situ to release the agent. For example, when the sugar is a glucuronide, glucuronidases, which are more active on the surface of cancer cells compared to normal cells, will cleave the glucuronide bond, thus releasing the agent. Thus the conjugates of the invention have enhanced cancer cell target specificity.

### SUMMARY OF THE INVENTION

The invention relates to conjugates comprising a cancer cell specific ligand, linked to a sugar, linked to a cancer chemotherapeutic agent or BNCT agent and uses thereof.

The present invention relates in particular to compounds of the Formula (I):
A-R′—X (I)
wherein A is the residue of a cancer chemotherapeutic agent or BNCT agent, R′ is a sugar residue, and X is a cancer cell specific ligand.

The invention also relates to a method for the treatment or amelioration of any condition treatable with cancer chemotherapeutic agents and BNCT agents, e.g. a hyperproliferative disease, comprising administering to an animal in need thereof, an effective amount of a compound of the invention, or a pharmaceutically acceptable salt thereof. In a preferred embodiment, the hyperproliferative disease is cancer.

The invention also relates to pharmaceutical compositions comprising the compounds of the invention and a pharmaceutically acceptable carrier.

The invention also relates to a method of preparing a compound of Formula (I) which comprises condensing a protected sugar with a cancer chemotherapeutic agent. The conjugate is then condensed with a cancer cell specific ligand and the protecting groups may then be partially or completely removed.

### DETAILED DESCRIPTION OF THE INVENTION

Cancer Cell Specific Ligands

These ligands include small organic molecules, carbohydrates and peptides that bind specifically to cancer cells. Preferably, the ligands are peptides, preferably 5-8 amino acids long within a peptide comprising 20 amino acids or less and, more preferably, are cyclic peptides of 20 amino acids or less and containing the ligand peptide of 5-8 amino acids. Also preferably, the ligand portion of the cyclic peptide are devoid of a lysine unit, eliminating the possibility that the side chain amine will interfere with the new amide bond formation with the sugar-agent/label conjugate. One such peptide which contains a lysine end unit bearing free amine ready for linkage to a pro-drug is ER Peptide 3: Acetyl-D F Q C P Y V E C V V N A P G G K-NH2 (SEQ ID NO:1). Preferably, the peptide is a cyclic peptide having a disulfide bond between the two cysteine groups. Another example is Asp-Phe-Gln-Cys-Pro-Tyr-Val-Glu-Cys-Val-Val-Asn-Ala-Pro-Gly-Gly-Lys-NH2 (SEQ ID NO:2).

uPA (urokine plasminogen activator), which converts plasminogen to plasmin, is more widely expressed in the cancer cells. U.S. Pat. No. 6,120,765 discloses uPA peptide fragments having more than 5 amino acids and less than 13 contiguous amino acids and bind to the urokinase plasminogen activator receptor (uPAR). These peptides have mitogenic action due to the agonist action of uPA. One such cyclic protein useful in the present invention is shown below and has a free carboxyl end for attachment to amine end of prodrugs. Any of the peptides described in U.S. Pat. No. 6,120,765, whether cyclic or straight chained, may be used in the practice of the invention.

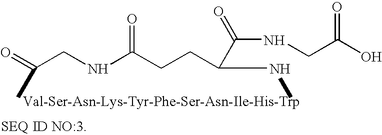

A synthetic variant useful in the practice of the present invention that accommodates attachment to carboxyl end of a glucuronate is also described in U.S. Pat. No. 6,120,765:

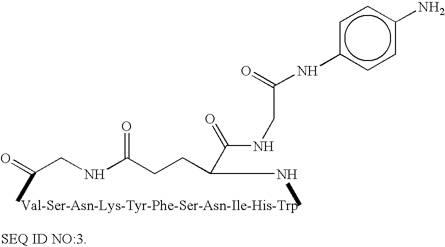

Other examples of peptides that may be used in the practice of the invention include the uPAR-targeting peptide of 11 amino acids; and substitution variants thereof, corresponding to human uPA(20-30), as described in U.S. Pat. No. 6,277,818.

Additional examples of peptides that may be used in the practice of the invention are described in U.S. Pat. No. 6,339,139 which describes peptide conjugates for targeting cancer cells. According to this patent, IGF-I R and IGF-II R are over-expressed in human hepatic cancer and other malignancies. EGF R is highly expressed in human hepatic, mammary, ovarian, gastric, cervix cancer, glioblastoma, lung adenocarcinoma, nasopharyngeal cancer etc. VEGF R is over-expressed in vascular endothelial cells of tumor blood vessels and some cancer cells.

Additional examples of peptides that may be used are disclosed in U.S. Pat. No. 6,087,109 which discloses ST receptor proteins. Such ST receptors are found on colorectal cells, including local and metastasized colorectal cancer cells. In normal human individuals, ST receptors are found exclusively in cells of intestine, in particular in cells in the duodenum, small intestine (jejunum and ileum), the large intestine, colon (cecum, ascending colon, transverse colon, descending colon and sigmoid colon) and rectum. Thus, conjugates of the present invention comprising peptide ligands for the ST receptor may be used to treat local and metastasized colorectal cancer.

Small peptides are especially preferred, as they are easy to use in organic synthesis for conjugation and targeting. Cyclic peptides are also useful since the shape of the cyclic peptides don't change or are perturbed by the neighboring pro-drug attachment.

Where the peptide contains an internal lysine, it is preferably protected prior to the amide bond formation with the pro-drug. Examples of lysine protecting groups include acetyl, t-butyloxy carbonyl (BOC) and benzyloxycarbonyl groups (carbamate protection). BOC and benzyloxycarbonyl groups can be deprotected with trifluroacetic acid or hydrogenation, respectively, depending upon compatibility with the rest of the molecule. The acetyl groups may be left without deprotection.

Other cancer cell specific ligands include those described in U.S. Pat. No. 6,322,770. The targeting moiety comprising an indazole binds to a receptor that is expressed in tumor neovasculature.

Additional cancer cell specific ligands are disclosed in U.S. Pat. No. 5,942,492, which discloses compounds having affinity for certain cancer cells, e.g. lung carcinomas, colon carcinomas, renal carcinomas, prostate carcinomas, breast carcinomas, malignant melanomas, gliomas, neuroblastomas and pheochromocytomas.

Other cancer cell specific ligands include monoclonal antibodies and fragments thereof that bind to cancer cell surface proteins, e.g. receptors. Examples of such antibodies include those specific for the extracellular domain of the EGF receptor as disclosed in U.S. Pat. No. 6,217,866.

Other cancer cell specific ligands include cyclic peptides which recognize glucuronidase, uPA, ER (see S. Sompuram, et al., Clin. Chem. 48: 410-420 (2002)), PR (progesterone receptor), and Her-2 receptors.

Cancer Chemotherapeutic Agents

Cancer chemotherapeutic agents useful in the present invention include cytotoxic compounds such as taxol, 5-fluorouracil, etoposide, mitomycin C, colchicine, vinblastine, paclitaxel, docetaxel, camptothecin, topotecan, doxorubicin, chlorozotocin, ranimustine, nimustine, etoposide, teniposide, perfosfamide, 5-azacytidine, 5-fluorouracil, methotrexate, 5-fluoro-2′-deoxy-uridine, vidarabine, hydroxyurea, thioguanine, melphalan, chlorambucil, vincristine, epirubicin, aclarubicin, bleomycin, mitoxantrone, elliptinium, fludarabine, retinoic acid, thalidomide and 4-hydroxy tamoxifen.

BNCT Agents

Such BNCT agents are organic molecules comprising boron. When administered to an animal, the cancer cell specific ligand will bind to the cancer cells, thus localizing the conjugate to the site of cancer. When irradiated with neutrons, for example, ¹⁰B atoms absorb the neutrons and release ⁷Li, gamma rays and α-particles which kill the tumor cells.

Specific examples of such boron-containing compounds that may be used in the practice of the invention include boronated nucleotide analogs (Lato, S. M. et al., Nucl. Acid. Res. 30:1401-7 (2002); Tjarks, W. et al., Nucleosides Nucleotides Nucl. Acids 20:695-8 (2001); U.S. Pat. No. 6,180,766), p-10 borophenylalanine (Dagrosa, M. A. et al., Thyroid 12:7-12 (2002); U.S. Pat. No. 6,169,076), boronated polylysine (Novick, S. et al., Nucl. Med. Biol. 29:159-67 (2002)), the unnatural amino acid 1-amino-3-[2-(1,7 dicarba-closo-dodecaboran-(12)-1-yl)ethyl]-cyclobutane (Srivastava, R. R. et al., J. Org. Chem. 13:4476-4478 (1997)), isocyanato dicarbaborane (Wu, Y. et al., Inorg. Chem. 36:4753-4761 (1997)), N-benzylpolyamines containing boron and fluorine (Martin, B. et al., J. Med. Chem. 44:3653-64 (2001)), the boronated dipeptide borotrimethylglycyl-phenylalanine (Takagaki, M. et al., Radiat. Res. 156:118-22 (2001), borylated derivatives of ferrrocenium (Weissfloch, L. et al., Biometals 14:43-9 (2001)), boron-containing hormone analogs which bind to hormone receptors (U.S. Pat. No. 6,074,625), and boron clusters conjugated to organic molecules containing hydroxy and amine groups (U.S. Pat. No. 6,074,625).

Sugar Residues

Sugar residues that are useful in the practice of the present invention include glucose, glucosamine, glucuronic acid, ribose, and the 2-deoxy derivatives thereof, e.g. 2-deoxy glucose, 2-deoxy-2-fluoro glucose and 2-deoxy ribose. In a preferred embodiment, the sugar residue is glucuronic acid which renders the conjugate more polar and water soluble, thus ameliorating any solubility problems of the cancer cell specific ligand and/or agent.

Other sugar residues that may be used in the practice of the invention include derivatives of glucose, glucosamine and glucuronic acid and their mono fluoro derivatives. Preferably, endogenous glucosidases, glucuronidases, and amidases will recognize and cleave the sugar derivative-agent bond, thus releasing the agent. In other embodiments, the derivative is chosen so that the sugar derivative-agent bond is not cleaved, thus retaining the agent on or in the cancer cells.

The sugar residues may have free hydroxy groups, or the hydroxy groups may be acylated, e.g. with a group R₄—C═O)—, wherein R₄ is hydrogen, C₁₋₆ alkyl, C₆₋₁₀ substituted or unsubstituted aryl or C₇₋₁₆ aralkyl. Preferably, the acyl groups are acetyl or propionyl. Other preferred R₄ groups are phenyl, nitrophenyl, halophenyl, lower alkyl substituted phenyl, lower alkoxy substituted phenyl and the like or benzyl, lower alkoxy substituted benzyl and the like.

The sugar residues may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

The compounds of the invention may be in the form of an acid/amine addition salt by treatment with an inorganic or organic acid/base.

Methods of Making the Compounds of the Invention

The cancer cell specific ligands may be conjugated to the cancer chemotherapeutic agent/BNCT agent by the addition of a sugar as described above. Bifunctional units like glucuronic acid and glucosamine are preferred as they are stable molecules and are known to be cleaved by glucuronidase and glucosidase enzymes. Glucuronidase enzymes are more prevalent and highly expressed in cancer cells over normal cells.

Amadori rearrangement is a main concern when linking sugars with peptides. This reaction is also called Malliard reaction. However, the blocking of the anomeric position of the sugar avoids this adverse reaction between the sugar and the peptide. The anomeric position may be blocked with the agent having a hydroxy function (e.g. alcohols/phenols/carboxyl groups/enols) present on the agent. The coupling of the agent with a cancer cell specific ligand such as a peptide can be carried out with reagents such as EDC and DMT-MM {4-(4,6-dimethoxy-1,3,5-triazin-2-yl)-4-methylmorpholinium chloride}. DMT-MM is more versatile than EDC, as the conjugation reaction may be carried out in protic solvents such as methanol, ethanol and water. Amide bond formation may be accomplished elegantly by this method.

Glucuronate/agent conjugates may be prepared by reacting protected glucuronic acid containing an activated anomeric position (e.g. the 1-halo, trimethylsilyl and tricholoroimidate derivatives) together with and the agent containing hydroxyl, phenolic and carboxyl functions. After the conjugation and deprotection, the carboxyl group of the glucuronate may be conjugated to the amine end of a cancer cell specific ligand such as a peptide. Similarly protected glucosamine may be conjugated and deprotected to give a conjugate having a free amine that may be conjugated to the C-terminal end of a peptide. The amide bond may be obtained by reaction with DMT-MM in a protic solvent such as methanol and water in which the peptide and glucuronates are soluble. Preferably, the cancer cell specific ligand is a small cyclic peptide, e.g. a 5-30 amino acid peptide.

In one particular embodiment, the agent may be conjugated to glucosamine as shown in Scheme 1:

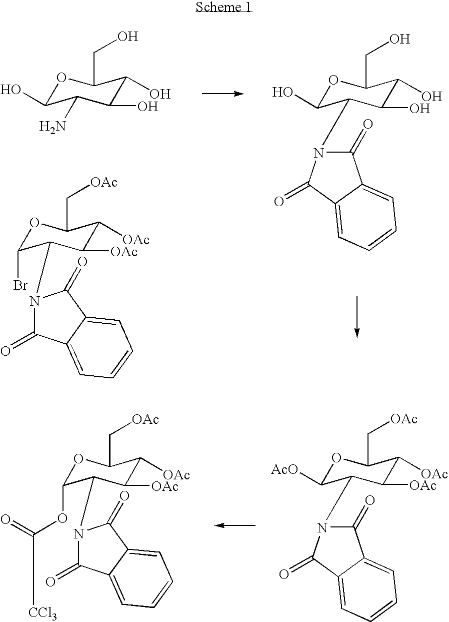

In place of phthalamide, the protecting group may also be the p-anisaldehyde imine which is easier to deprotect under mildly acidic conditions.

After introducing the sugar residue to the drug, amide formation reaction is carried out with a cancer cell specific ligand, in particular, peptides chosen from cyclic peptides of uPA-agonists. This reaction is carried out after deprotection and reacting either the amine or acid of the sugar with DMT-MM and the peptide that has an exposed carboxyl or amine function, respectively.

A number of sugar-chemotherapeutic agent conjugates are shown in Scheme 2.

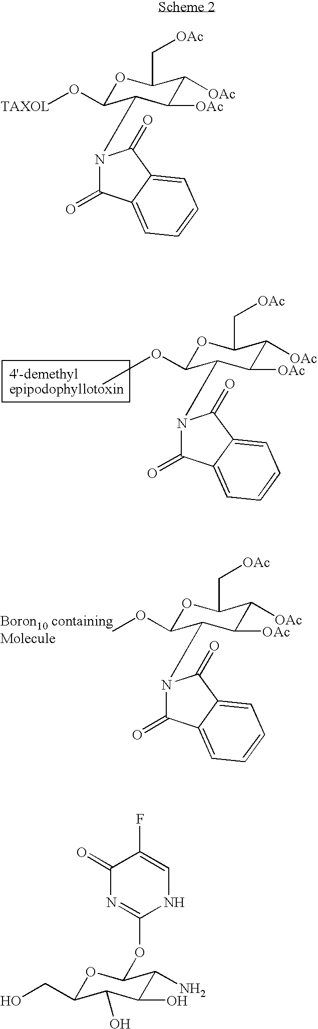

Similarly, glucuronide conjugates may be prepared as shown in Scheme 3.

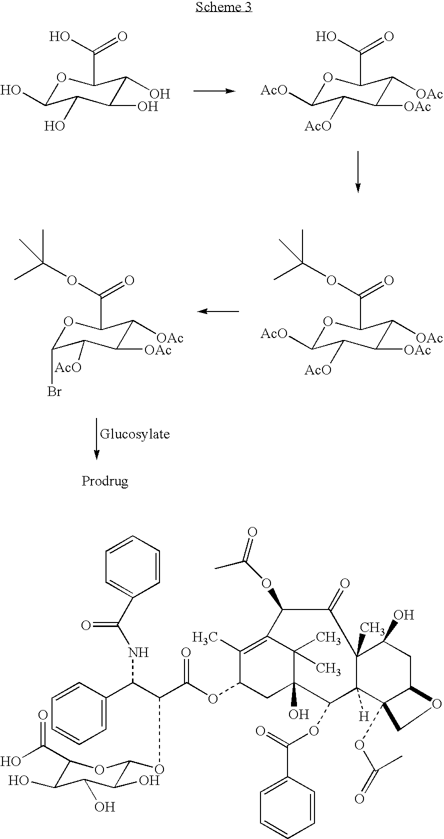

Other glucuronide conjugates are shown in Scheme 4.

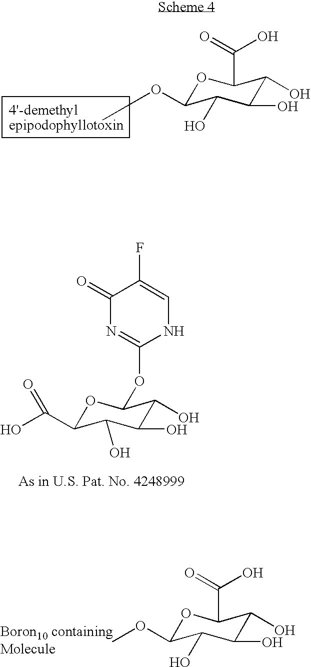

Examples of conjugates comprising uPA derived cyclic peptide and various sugars and cancer chemotherapeutic agents include:

5-Flurouracil (SEQ ID NO:3 for each sequence):

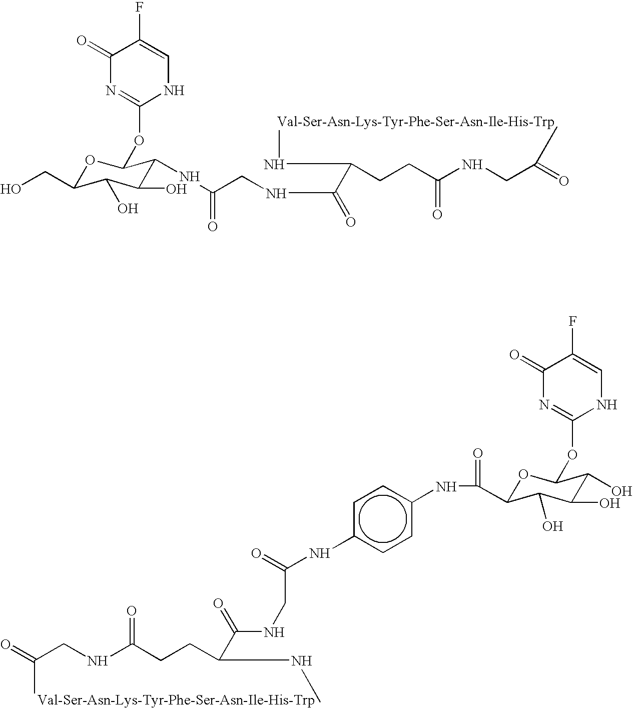

Etoposide (SEQ ID NO:3 for each sequence):

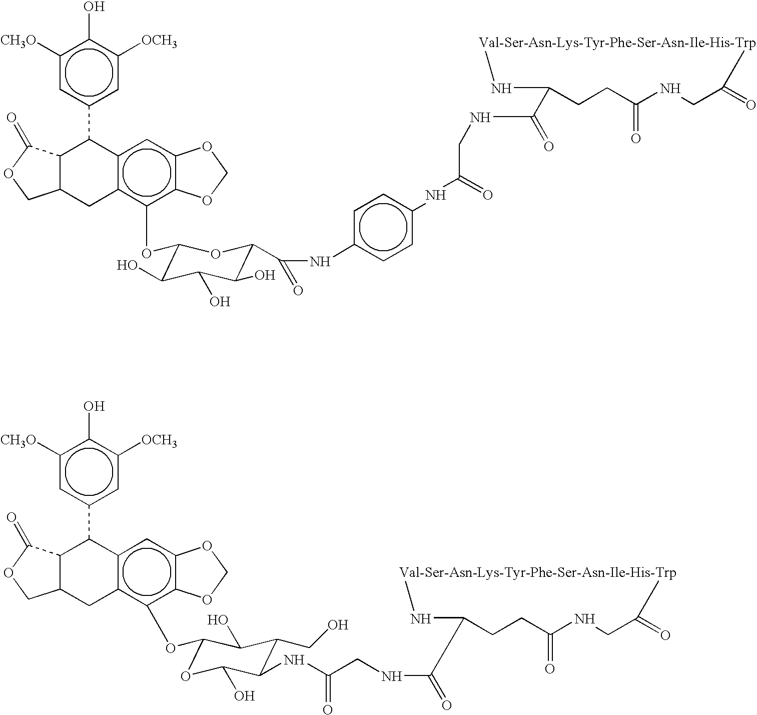

Taxol (SEQ ID NO:3 for each sequence):

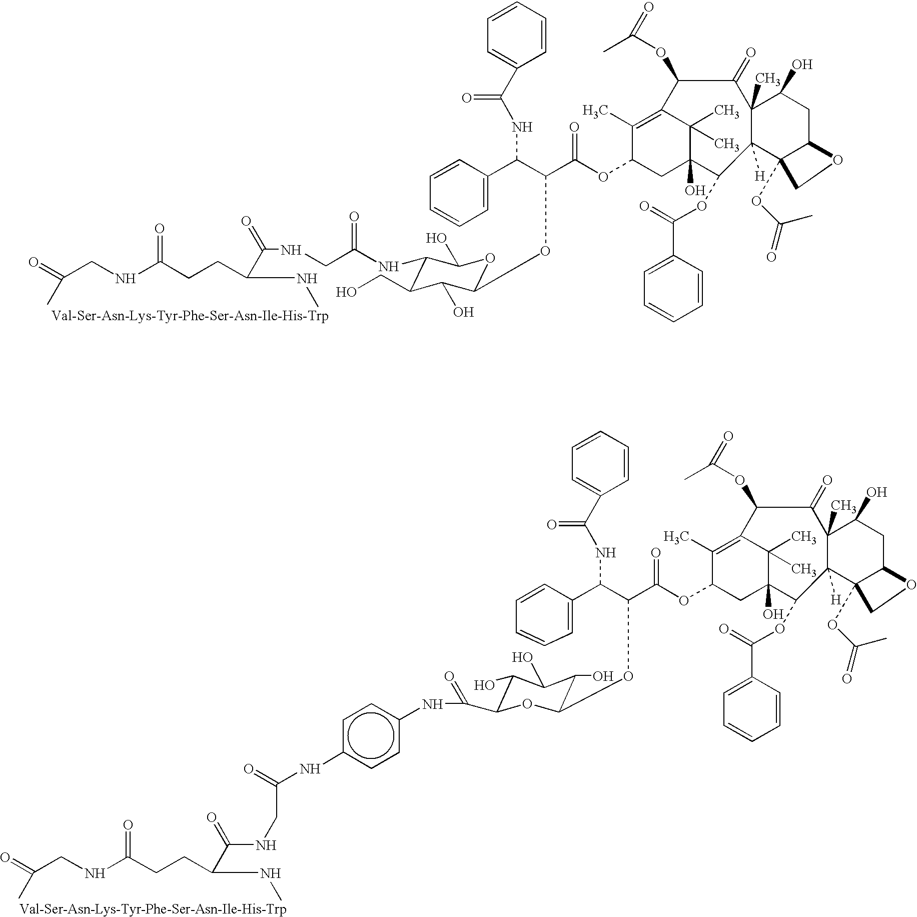

Conjugates comprising BNCT agents may be prepared as shown in Scheme 4. See also U.S. Pat. No. 6,074,625 which describes boron-containing hormone analogs.

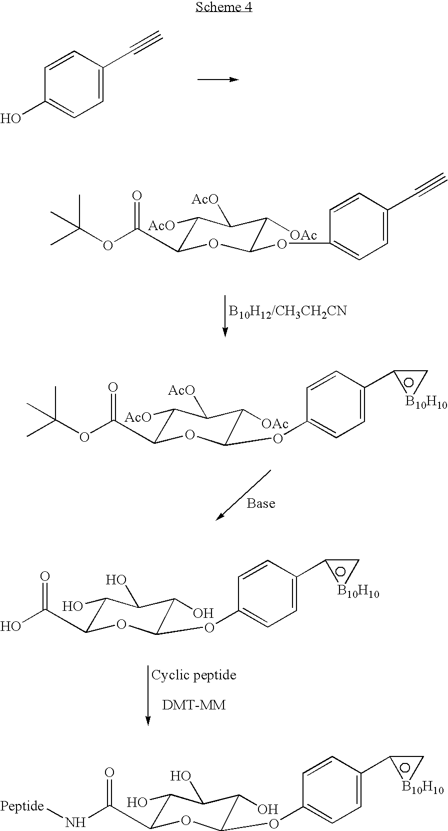

Particular examples of such conjugates include (SEQ ID NO:3 for each sequence):

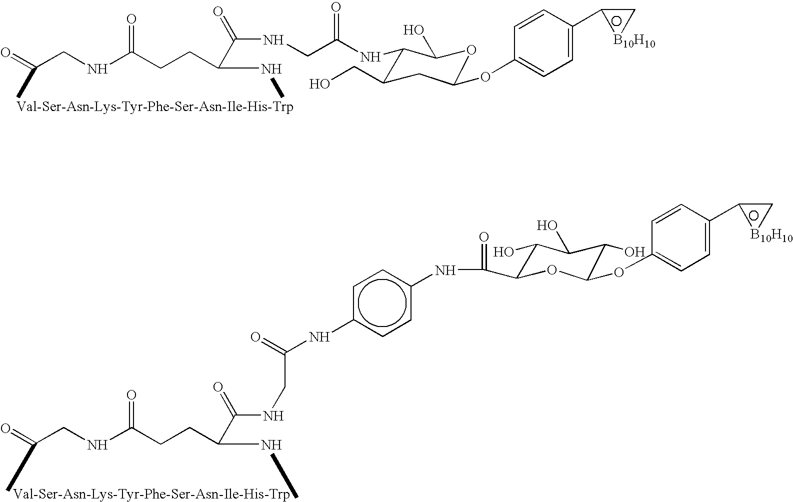

The amide bond formation can be prepared after de-protection and reacting either the amine or acid of the sugar with DMT-MM and the peptide that has an exposed carboxyl or amine function, respectively.

Methods of Use and Formulation

Particularly preferred routes of administration of the compounds of the present invention are per os, such as elixirs, tablets and capsules, as exemplified below, and by i.v. administration.

More generally, the compounds of the present invention can be administered in any appropriate pharmaceutically acceptable carrier for oral administration since the compounds are biologically active upon oral administration. The compounds of the invention may also be administered in any appropriate pharmaceutical carrier for parenteral, intramuscular, transdermal, intranasal, buccal or inhalation administration. They can be administered by any means that treat or ameliorate the conditions and diseases described herein.

The dosage administered will depend on the age, health and weight of the recipient, kind of concurrent treatment, if any, frequency of treatment and the nature of the effect desired. An exemplary systemic daily dosage is about 0.1 mg to about 500 mg. Normally, from about 1.0 mg to 100 mg daily of the compounds, in one or more dosages per day, is effective to obtain the desired results. One of ordinary skill in the art can determine the optimal dosages and concentrations of active compounds with only routine experimentation.

The compounds can be employed in dosage forms such as tablets and capsules for oral administration. Such dosage forms may comprise well known pharmaceutically acceptable carriers and excipients. In a preferred embodiment, the dosage forms comprise cyclodextran and/or other saccharides and/or sugar alcohols. The compounds may also be formulated in a sterile liquid for formulations such as solutions (e.g. in saline) or suspensions for parenteral use. A lipid vehicle can be used in parenteral administration.

The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Remington's Pharmaceutical Sciences, 18^{th} Edition, Gennaro et al. (eds.), 1990, exemplifies methods of preparing pharmaceutical compositions.

The compounds may also be employed in fast dissolving dosage forms, as described in U.S. Pat. No. 6,316,027, comprising the compounds of the invention, water, gelatin and other ingredients.

The compounds of the invention may be formulated as part of a liposomal composition.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, drops or transdermal patch. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight of the active compounds.

The compounds of the invention are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, veterinary animals and pets, although the invention is not intended to be so limited.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

1. A compound comprising a cancer cell specific ligand, linked to a sugar, linked to a cancer chemotherapeutic agent or boron neutron capture therapy (BNCT) agent.

2. The compound of claim 1, wherein said cancer cell specific ligand is a peptide.

3. The compound of claim 2, wherein said peptide is a cyclic peptide of 20 amino acids or less and containing the ligand peptide of 5-8 amino acids.

4. The compound of claim 1, wherein said sugar is glucose, glucosamine, glucuronic acid, ribose, or the 2-deoxy derivatives thereof.

5. The compound of claim 1, wherein said agent is taxol, 5-fluorouracil or etoposide.

6. The compound of claim 1, wherein said agent is a boron neutron capture therapy (BNCT) agent.

7. The compound of claim 1, wherein said compound has the formula (SEQ ID NO:3 for each sequence):

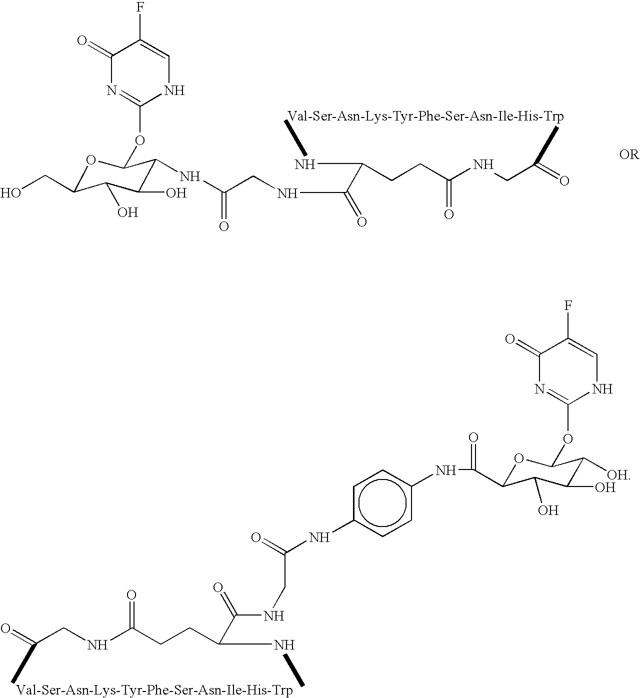

8. The compound of claim 1, wherein said compound has the formula (SEQ ID NO:3 for each sequence):

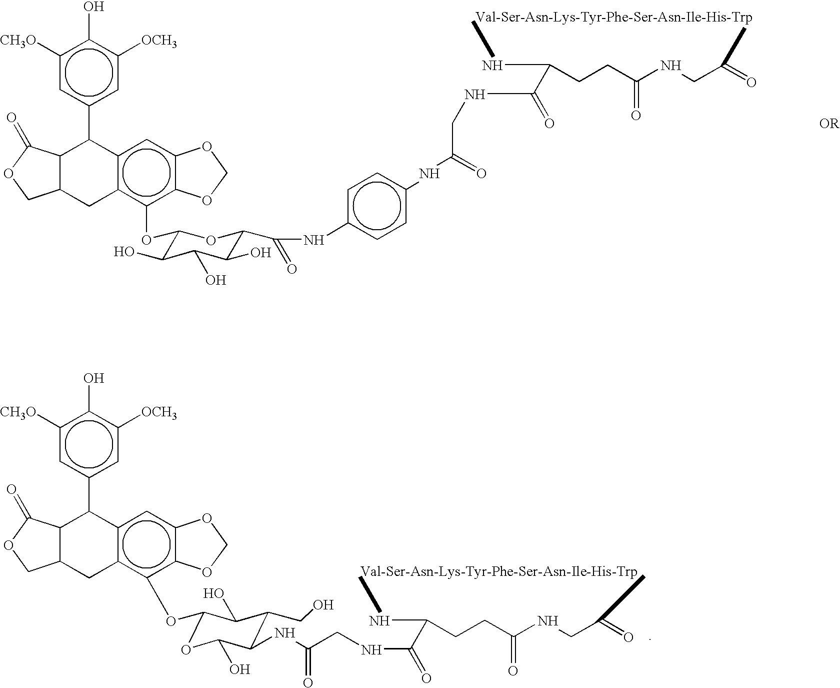

9. The compound of claim 1, which has the formula (SEQ ID NO:3 for each sequence):

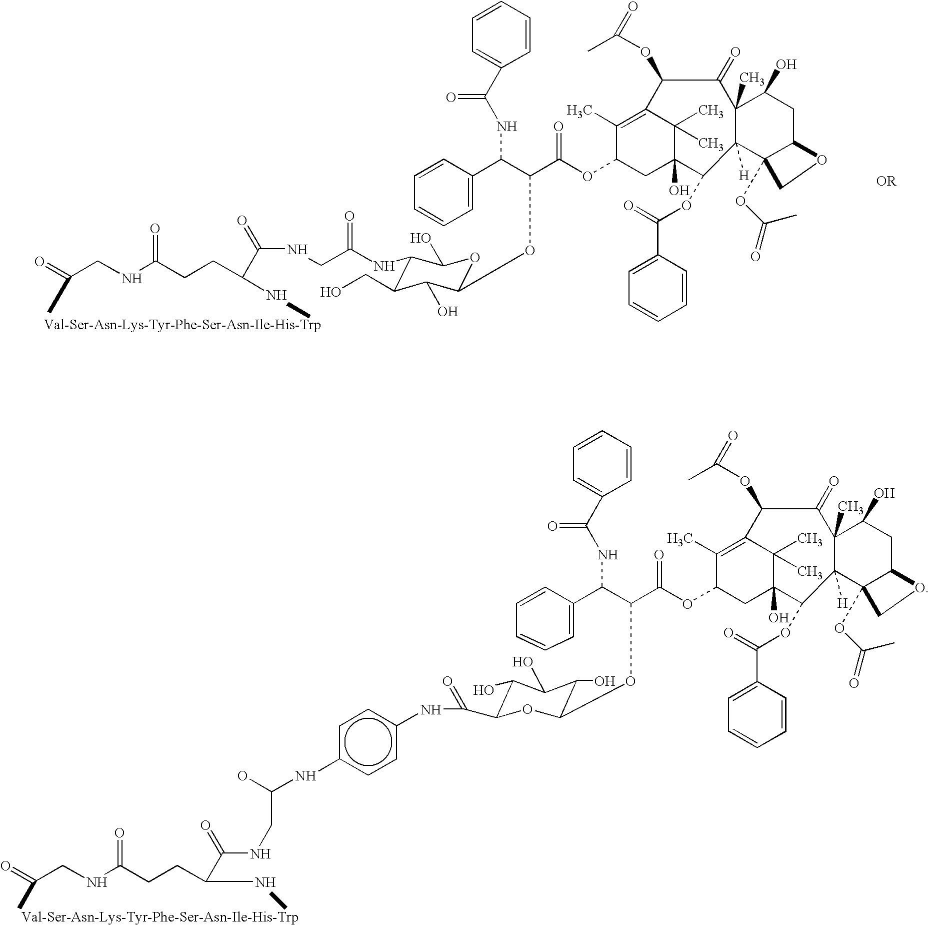

10. The compound of claim 1, wherein said compound has the formula (SEQ ID NO:3 for each sequence):

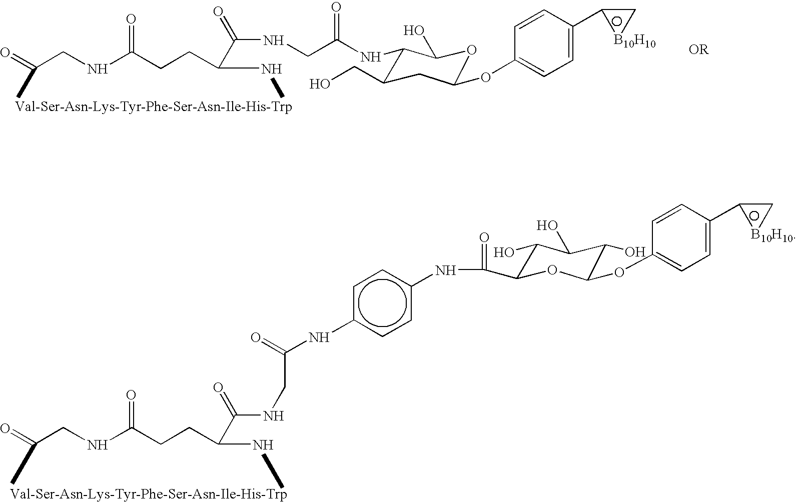

11. A pharmaceutical composition comprising the compound of claim 1 and a pharmaceutically acceptable carrier.

12. A method for the treatment or amelioration of cancer in an animal, comprising administering to an animal in need thereof an effective amount of the compound of claim 1.

13. The method of claim 12, wherein said compound is administered as part of a pharmaceutical composition comprising a pharmaceutically acceptable carrier therefor.

14. The method of claim 12, wherein said animal is a human.

15. The method of claim 12, wherein said agent is a BNCT agent and said method further comprises irradiating said animal with neutrons.


SEQUENCE LISTING (3 sequences)

SEQ ID NO: 1 - protein, 17 aa, Artificial sequence
  ER Peptide 3
DFQCPYVECVVNAPGGK

SEQ ID NO: 2 - protein, 17 aa, Artificial sequence
  Synthetic Peptide
DFQCPYVECVVNAPGGK

SEQ ID NO: 3 - protein, 11 aa, Artificial sequence
  Synthetic Peptide
VSNKYFSNIHW